(19) United States
(12) Patent Application Publication
(10) Pub. No.: US 2006/0195062 A1
(43) Pub. Date: Aug. 31, 2006

(54) Apparatus for locking concentric components in alignment

(76) Inventor: Robert F. Gremel, Huntington Beach, CA (US)

Correspondence Address: WALTER A. HACKLER, Ph.D.;PATENT LAW OFFICE, SUITE B, 2372 S.E. BRISTOL STREET, NEWPORT BEACH, CA, 92660-0755, US

(21) Appl. No.: 11/066,774

(22) Filed: Feb. 25, 2005

(51) Int. Cl. A61M 5/00; A61M 5/32

(52) U.S. Cl. 604/110; 604/192

(57) ABSTRACT

Apparatus for locking concentric components and alignment with one another generally includes a syringe sheath having a cylindrical wall between a bottom and an open top. A syringe body includes a bottom thereabout for sliding engagement with a syringe sheath inner surface and cleats formed in the syringe body wall in an opposing relationship are guided in two channels formed in the syringe sheath wall. Each of the cleats includes one end attached to the syringe body wall, a free end and a body portion disposed parallel to the channels. The free ends are spaced apart from the syringe body wall and engagement of the cleats with shoulders limits insertion of the syringe body into the syringe sheath. Placement of the cleats prevents tilting of the syringe body from releasing the cleats from corresponding shoulders.

The present invention generally relates to apparatus for locking concentric components in alignment with one another and is more particularly directed to a shielded blood collection tube holder (BCTH) having an outer protective shield and a retractable needle and sheath assembly.

Single-use disposable blood collection devices are used in much the same manner that a single-use disposable sterile hypodermic syringe needles are used.

Conventional hospital and clinical laboratory procedures often include the collection of multiple samples of blood from a patient in a single medical procedure. This procedure involves the use of a blood collection tube holder, sterile needle, and one or more blood collection tubes.

In use, the blood collection tubes have rubber stoppers and have a vacuum inside, and the blood collection needle is releasably attached to the blood collection tube holder. The blood collection needle has a point at each end, one for the patient, and the other to penetrate the rubber stopper of the collection tube. The blood collection needle is inserted into a patient's vein for withdrawal of blood, which is introduced into the blood collection tube. The vacuum within the blood collection tube provides suction to withdraw the blood from the patient. Afterward, the blood collection tube is removed and any number of additional tubes may be inserted into the holder and filled with blood.

Upon completion of blood collection, the needle is removed from the patient. Unfortunately, health care workers are susceptible to needle sticks either due to careless handling or during disposal of the needle after use.

In order to prevent such accidental needle contact, shielded safety syringes having retractable needles have been developed see for example U.S. Pat. No. 4,813,426. However, such devices do not provide for insuring that once the needle is retracted that it cannot be again extended.

Present invention provides apparatus for locking concentric parts, such as a sheath and a blood collection tube holder body in alignment with one another, such that once a the needle is withdrawn into the sheath, it cannot be re-extended despite manipulation of the tube holder, which is extended from the sheath when the needle is disposed within the sheath.

### SUMMARY OF THE INVENTION

Apparatus in accordance with the present invention for locking concentric parts in alignment with one another generally includes an outer component, which may be a sheath, having a cylindrical wall between a bottom and an open top.

Two channels formed in the outer component wall in a 180° spaced apart relationship with one another around an inner surface of the outer component wall are parallel with a longitudinal axis of the outer component and have a first shoulder disposed proximate the outer component top.

An inner component, such as, for example, a blood collection tube holder body, includes a bottom with a ring thereabout for sliding engagement with the outer component inner surface.

Two cleats, formed in the inner component wall in an opposing relationship, are provided with each cleat having one end attached to the inner component outer wall, a free end and a body portion disposed parallel to the longitudinal axis.

The free ends are spaced apart from the inner component outer wall and each cleat is sized for sliding in one of the channels of the outer component and engaging the first shoulder. Engagement of the cleats with the first shoulder limits insertion of the inner component into the outer component at a first position with a portion of the inner component wall extending outwardly from the outer component open top.

Means are provided which includes the opposing relationship between the cleats, for preventing releasing of both cleats from the first shoulder by tilting of the inner component extended portion. Accordingly, abusive handling of the extended portion of the inner component, for example, BCTH body, cannot inadvertently dislodge both cleats from the channel/shoulder.

Thus, it is impossible to re-extend a needle attached to the syringe body by tilting away from one cleat and weakening its ability to retain the outer component or sheath. That is, any tilting toward one cleat simultaneously improves an opposing cleat holding power.

Each channel in one embodiment may include a second shoulder disposed proximate the outer component bottom for engaging the cleats and eliminating insertion of the inner component into the outer component. At this second position, the inner component inner connecting wall is mostly within the outer component. At this arrangement, a needle, removably attached to the inner component, or BCTH body, extends outwardly from the outer component or sheath.

A spring may be provided and disposed between the inner component bottom and the outer component top in order to bias the inner and outer components apart from one another for automatic retraction of a needle extending from the outer component bottom.

A latch is formed in the outer surface of the outer component wall proximate the outer top for engaging the inner component top to prevent spring expansion and movement of the inner component to the first position. In this position, the needle is extended and is locked in such extended position by the latch.

In addition, a retaining tab may be disposed in an outer surface of the outer component wall proximate the open top. A tab includes one end attached to the wall outer surface, a free end and a body disposed parallel to the longitudinal axis. The body has suitable thickness for enabling depression of the free end towards the wall inner surface and a slot formed in the outer component wall beneath the tab. The slot includes an end sized and disposed for enabling the free end to be forced therepast by depression of the tab in order to retain the free end inside the outer component for engagement with the ring, thus preventing separation of the inner component from the outer component.

Thus, when retracted, the inner component, or BCTH body, and needle is prevented from movement in either axial direction within the outer component, or syringe sheath, thus positively locking the needle within the sheath.

### BRIEF DESCRIPTION OF THE DRAWINGS

The advantages and features of the present invention will be better understood by the following description when considered in conjunction with the accompanying drawings in which:

FIG. 1 is a cross sectional view of apparatus in accordance with the present invention for locking concentric parts in alignment with one another and generally showing a body with a needle removably attached thereto, a sheath along with a spring and a blood collection tube;

FIG. 2 is a cross sectional view similar to that shown in FIG. 1 with the spring compressed and the needle extended outside of the syringe sheath;

FIG. 3 is a cross sectional view taken along the line 3-3 of FIG. 2, more clearly showing a latch for holding the syringe body within the syringe sheath with the needle extended therefrom along with a retaining tab for providing locking of the syringe body and syringe sheath, as will be hereinafter described in greater detail;

FIG. 4 is an enlarged view of the tab shown in FIG. 3 before the depression toward the cylindrical wall;

FIG. 5 is a tab, as shown in FIG. 4, but depressed with an end thereof held within a wall of the syringe sheath for engaging a syringe body; and

FIG. 6 is an enlarge view of the tab as it engages a ring on the syringe body for locking to the syringe sheath.

### DETAILED DESCRIPTION

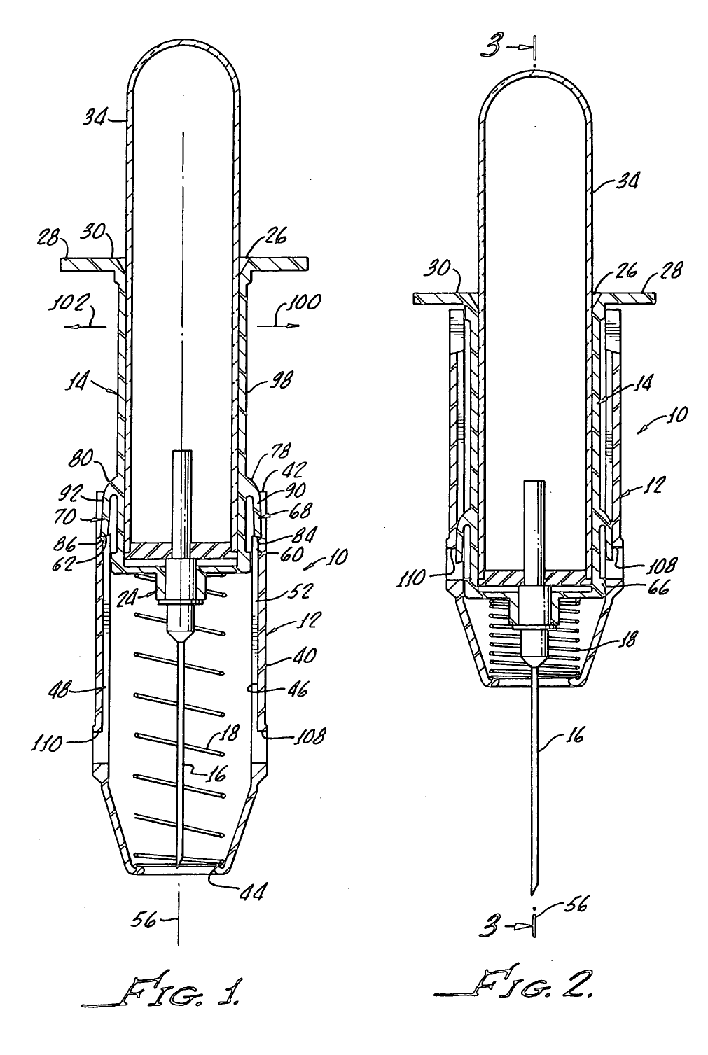

With reference to FIGS. 1 and 2, there is shown apparatus 10 in accordance with the present invention, which generally includes an outer component, or sheath 12 and a syringe body 14, along with a needle 16 removably attached to a bottom 24 of the body 14.

The body 14 includes a top 26 with a flange 28 and an opening 30 sized for receiving a blood collection tube 34 in a conventional manner.

The sheath 12 and body 14 are preferably molded from a suitable material for syringe application and the present invention provides for an inexpensive manufacture of the apparatus 10.

The sheath 12 includes a cylindrical wall 40 extending between an open top 42 and an open bottom 44. Two channels 46, 48 are formed in the sheath wall 40 in a 180° spaced apart relationship to one another around an inner surface 52 of the wall 40. The channels 46, 48 are parallel with a longitudinal axis 56 and each of the channels 46, 48 include first shoulders 60, 62 respectively proximate the sheath top 42.

The body bottom includes a ring 66, sized for sliding engagement with the sheath inner surface 52.

Cleats 68, 70 are formed in a wall of the body in an opposing relationship. Each cleat 68, 70 has one end 78, 80 attached to the sheath wall 72, a free end 84, 86, and a body portion 90, 92 disposed parallel to the longitudinal axis 56. As shown, the free ends 84, 86 are in a spaced apart relationship with the body wall 72.

Each cleat 68,70 is sized for sliding in one of the channels 46, 48. A design constraint of the cleats 68,70 is that they must be tall enough with respect to the sheath 12 to engage and lock, as hereinafter described, but also must be short enough with respect to the sheath 12 that they do not generate excessive friction to limit motion of the body 14 within the sheath 12. The result of this compromise is a delicate balance to achieve all the required functionalities, and thus is a dimensionally sensitive design.

For example, with a sheath diameter of approximately 0.86 inch, the channel may have a depth of about 0.040 inches, with the cleat 68,70 length may be approximately 0.385 inches, thickness of about 0.040 inches.

These dimensions enable the locking feature of the present invention while not providing excessive friction for movement of the body 14 within the sheath 12.

As shown in FIG. 1, engagement of the cleats 68 with the first shoulders 60,62 limits insertion of the syringe body 14 into the syringe sheath 12 at a first position, as shown in FIG. 1, with a portion 98 of the body 14 extending outwardly from the sheath top 42.

The opposing relationship between the cleats 68,70 provides a means for preventing tilting of the syringe body 14, as indicated by the arrows 100, 102 from releasing both cleats 68,70 from the first shoulders 60,62. Thus, this arrangement of cleats makes it impossible to re-extend the needle from the first position by tilting as shown by the arrows 100,102, since the lifting of the cleat 68 from the first shoulder 60, thereby weakening its ability to retain the body 14, concomitantly causes the cleat 70 to dig in to the shoulder 72, thus increasing the holding power between the cleat 70 and shoulder 62.

Shown in the first position in FIG. 1, the needle 16 is totally within the sheath 12 and is moved to this position after use thus, mandating that the needle 16 cannot be re-extended.

Each of the channels 46,48 may optimally include a second shoulder 108,110 disposed proximate the sheath bottom 44 for engaging the cleat 68,70 and limiting insertion of the body 14 into the sheath 12 at a second position, (shown in FIG. 2) with the body 14 totally within the sheath 12 and the needle 16 extending therefrom. The body 12 and sheath 14 are locked in this position via a latch 112 formed in an outer surface of the sheath 12 proximate the sheath top 42 for engaging the top 26 of the body 12 by capturing the flange 28. The latch then prevents the spring 18 from expanding and the moving syringe body 14 to the first position as shown in FIG. 1.

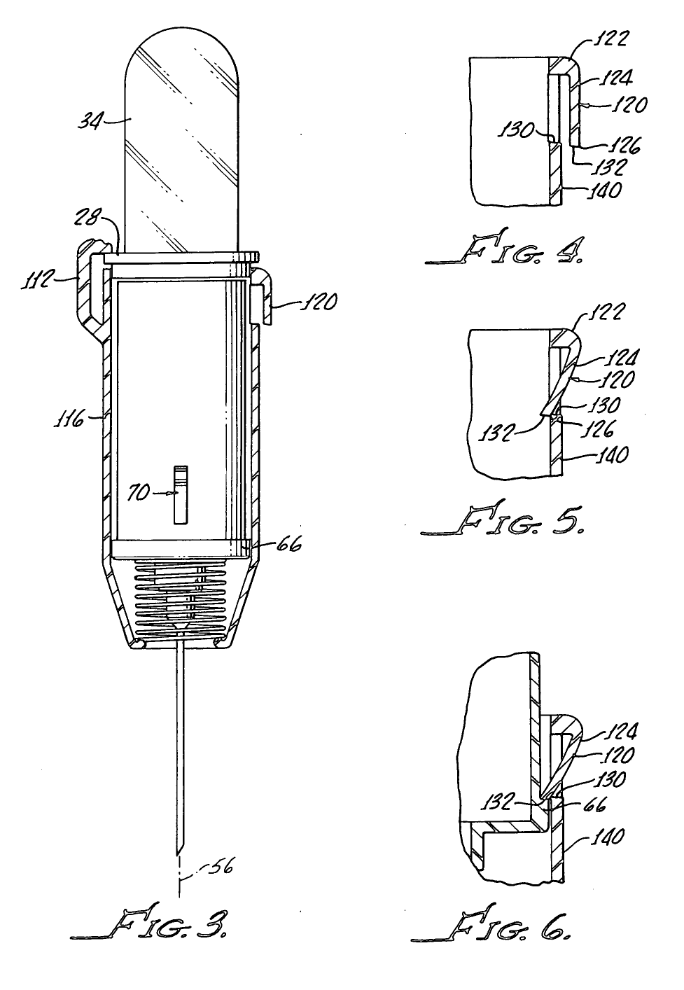

With reference to FIGS. 3-6, the apparatus 10 may further include a retaining tab 120.

Preferably, the tab 120 includes one end 122 molded into the sheath wall surface 116 and has a generally rectangular body 124 and a free end 126. The body 124 may be rectangular in shape for facilitating molding and has a suitable thickness for enabling depression of the free end 126 toward the surface 116.

A slot 128 formed in the wall 116 beneath the tab 120 may be formed with an end 130 sized and disposed for enabling the free end 126 to be forced therepast by depression of the tab 120 in order to retain the free end 126 inside the wall 40, as shown in FIG. 5. This, prevents separation of the body 14 from the sheath 12, as shown in FIG. 6.

As shown in FIGS. 3-6, the retaining tab 120 extends downwardly from the open top 42 generally parallel to the longitudinal axis 56 with the free end 126 disposed farther from the open end 42 then the end 122. In addition, the free end 126 may have a tapered engagement surface 132 for facilitating and enabling the free end 126 to be forced past the slot end 130.

The retaining tab 120 is disposed relative to the cleats 68,70 for preventing any relative motion between the syringe body 14 and the sheath 12 after movement of the syringe body 14 to the first position, as shown in FIG. 1. That is, in the position of FIG. 1, the tab 120 prevents removal of the syringe body 14 from the sheath 40 and the cleats 68,70 prevent insertion of the body 14 into the sheath 12. Thus, the needle 16 is permanently fixed within the sheath 12 after use of the needle and movement of the body 14 to the first position.

Although there has been hereinabove described a specific apparatus for locking concentric components in alignment in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any element, which is not specifically disclose herein. Accordingly, any and all modifications, variations or equivalent arrangements, which may occur to those skilled in the art, should be considered to be within the scope of the present invention as defined in the appended claims.

What is claimed is:

1. Apparatus for locking concentric parts in alignment with one another, said apparatus comprising:
an outer component having a cylindrical wall between a bottom and an open top;
two channels formed in the outer component wall in a 180° spaced about relationship with one another around an inner surface of the outer component wall, each channel being parallel with a longitudinal axis of said outer component and having a first shoulder disposed proximate the outer component top;
an inner component having a bottom with a ring thereabout for sliding engagement with the outer component inner surface, a top and an interconnecting wall;
two cleats formed in the inner component wall in an opposing relationship, each cleat having one end attached to the inner component wall, a free end and a body portion disposed parallel to the longitudinal axis, the free ends being spaced apart from the inner component wall, each cleat being sized for sliding in one of the channels and engaging the first shoulder, engagement of the cleats with the first shoulder limiting insertion of said inner component into said outer component at a first position with a portion of the inner component wall extending outwardly from the outer component open top, the opposing relationship between the cleats providing means for preventing tilting of the inner component extending portion from releasing both cleats from the first shoulder.

2. The apparatus according to claim 1 wherein each channel includes a second shoulder disposed proximate the outer component bottom for engaging said cleats and limiting insertion of said inner component into said outer component at a second portion with the inner component inner-connecting wall totally within said outer component.

3. The apparatus according to claim 2 further comprising a spring disposed between the inner component bottom and the outer component bottom.

4. The apparatus according to claim 3 further comprising a latch formed in an outer surface of the outer component wall proximate the outer component top for engaging the inner component top to prevent spring expansion and movement of said inner component to the first position.

5. The apparatus according to claim 4 further comprising a retaining tab disposed in outer surface of the outer component wall proximate the open top, the tab having one end attached to the wall outer surface, a free end and a body disposed parallel to the longitudinal axis, said tab having suitable thickness for enabling depression of said free end toward the wall outer surface and a slot formed in the outer component wall beneath the tab, said slot having an end sized and disposed for enabling said free end to be forced therepast, by depression of the tab, in order to retain said free end inside the outer component wall for engagement with said ring thus preventing separation of said inner component from said outer component.

6. The apparatus according to claim 5 further comprising a needle removably fixed to the inner component bottom, said needle being disposed within said outer component with the inner component to the first position and extending from said outer component with the inner component at the second position.

7. The apparatus according to claim 6 wherein said retaining tab is disposed at a position relative to the cleats for preventing any relative motion between said inner component and said outer component after movement of said inner component to the first position.

8. Apparatus for locking concentric parts in alignment with one another, said apparatus comprising:
a syringe sheath having a cylindrical wall between a bottom and an open top;
two channels formed in the syringe sheath wall in a 180° spaced about relationship with one another around an inner surface of the syringe sheath wall, each channel being parallel with a longitudinal axis of said syringe sheath and having a first shoulder disposed proximate the syringe sheath top;
a syringe body having a bottom with a ring thereabout for sliding engagement with the syringe sheath inner surface, a top and an interconnecting wall;
two cleats formed in the syringe body wall in an opposing relationship, each cleat having one end attached to the syringe body wall, a free end and a body portion disposed parallel to the longitudinal axis, the free ends being spaced apart from the syringe body wall, each cleat being sized for sliding in one of the channels and engaging the first shoulder, engagement of the cleats with the first shoulder limiting insertion of said syringe body into said syringe sheath at a first position with a portion of the syringe body wall extending outwardly from the syringe sheath open top, the opposing relationship between the cleats providing means for preventing tilting of the syringe body extending portion from releasing both cleats from the first shoulder.

9. The apparatus according to claim 8 wherein each channel includes a second shoulder disposed proximate the syringe sheath bottom for engaging said cleats and limiting insertion of said syringe body into said syringe sheath at a second portion with the syringe body inner-connecting wall totally within said syringe sheath.

10. The apparatus according to claim 9 further comprising a spring disposed between the syringe body bottom and the syringe sheath bottom.

11. The apparatus according to claim 10 further comprising a latch formed in an outer surface of the syringe sheath wall proximate the syringe sheath top for engaging the syringe body top to prevent spring expansion and movement of said syringe body to the first position.

12. The apparatus according to claim 11 further comprising a retaining tab disposed in outer surface of the syringe sheath wall proximate the open top, the tab having one end attached to the wall outer surface, a free end and a body disposed parallel to the longitudinal axis, said tab having suitable thickness for enabling depression of said free end toward the wall outer surface and a slot formed in the syringe sheath wall beneath the tab, said slot having an end sized and disposed for enabling said free end to be forced therepast, by depression of the tab, in order to retain said free end inside the syringe sheath wall for engagement with said ring thus preventing separation of said syringe body from said syringe sheath.

13. The apparatus according to claim 12 further comprising a needle removably fixed to the inner component bottom, said needle being disposed within said syringe sheath with the syringe body to the first position and extending from said syringe sheath with the syringe body at the second position.

14. The apparatus according to claim 13 wherein said retaining tab is disposed at a position relative to the cleats for preventing any relative motion between said syringe body and said syringe sheath after movement of said syringe body to the first position.

15. The apparatus according to claim 14 wherein the syringe body open top is sized for receiving a blood collection tube.
